(19) United States
(12) Patent Application Publication
(10) Pub. No.: US 2016/0106906 A1
(43) Pub. Date: Apr. 21, 2016

(54) BLOOD PUMP

(71) Applicant: QUANTA FLUID SOLUTIONS LTD, Alcester, Warwickshire (GB)

(72) Inventor: Clive Henry Buckberry, Warwick, Warwickshire (GB)

(21) Appl. No.: 14/779,765

(22) PCT Filed: Mar. 28, 2014
(86) PCT No.: PCT/GB2014/051007

§ 371 Date: Sep. 24, 2015

(30) Foreign Application Priority Data
Mar. 28, 2013 (GB) 1305758.3

(51) Int. Cl. A61M 1/36; F04B 43/02

(52) U.S. Cl. CPC A61M 1/3621; F04B 43/026; A61M 2205/121; A61M 2205/3331; A61M 2230/04

(57) ABSTRACT

The present invention defines a blood pump comprising a cartridge, the cartridge comprising a first recess therein, said first recess having a surface, and a flexible diaphragm closing said first recess, the first recess and the flexible diaphragm defining a first pump chamber, said first pump chamber having an inlet and an outlet wherein the flexible diaphragm of the first pump chamber is movable between a first position, separated in use from the surface of the first recess, wherein in said first position said first pump chamber has a maximum volume, and a second position, substantially adjacent to the surface of the first recess, wherein in said second position said first pump chamber has a minimum volume, a pump driver arranged to interface with the cartridge, said pump driver operable to move the flexible diaphragm of the first pump chamber in a first direction into said first recess to, in use, pump blood from the chamber and to move the flexible diaphragm of the first pump chamber in a second direction away from the first recess to, in use, draw blood into said first pump chamber, wherein, the cartridge further comprises one or more sensor cavities defined by respective recesses in the cartridge, the, or each, recess being closed by a flexible diaphragm.

The present invention relates to extracorporeal blood pumps, in particular, but not exclusively, to low haemolysis extracorporeal blood pumps.

Blood pumps for the extracorporeal circulation of blood are used in a number of medical applications, for example in hemodialysis. Hemodialysis machines are large expensive machines which a patient typically attends a medical facility to use. Due to the risk of cross contamination of one patients blood with another it is desirable to dispose of blood pumps after just one use.

Some hemodialysis machines are suitable for use by a patient at home. In such machines it is still desirable to dispose of the blood pump after a single use to prevent clotted blood from a previous dialysis session re-entering the patients body.

Conventionally, blood pumps are peristaltic pumps which are considered to be too expensive to dispose of after a single use.

Use of a membrane pump as a blood pump has provided a relatively cost effective means of providing single use blood pumps. Until now, such blood pumps have comprised a multi-part moulding, usually a pump part and a sensing part. Each part of a moulding carries a risk of errors in the moulding process which could have a negative effect on a patients blood being pumped through the blood pump. For example, moulding errors at flow ports in the blood pump can lead to stagnation of blood and ultimately clotting of the blood.

It is the purpose of the present invention to mitigate the above problems and to produce a blood pump that comprises a single moulding.

An aspect of the invention provides a blood pump comprising a cartridge, the cartridge comprising a first recess therein, said first recess having a surface, and a flexible diaphragm closing said first recess, the first recess and the flexible diaphragm defining a first pump chamber, said first pump chamber having an inlet and an outlet wherein the flexible diaphragm of the first pump chamber is movable between a first position, separated in use from the surface of the first recess, wherein in said first position said first pump chamber has a maximum volume, and a second position, substantially adjacent to the surface of the first recess, wherein in said second position said first pump chamber has a minimum volume, a pump driver arranged to interface with the cartridge, said pump driver operable to move the flexible diaphragm of the first pump chamber in a first direction into said first recess to, in use, pump blood from the chamber and to move the flexible diaphragm of the first pump chamber in a second direction away from the first recess to, in use, draw blood into said first pump chamber, wherein, the cartridge further comprises one or more sensor cavities defined by respective recesses in the cartridge, the, or each, recess being closed by a flexible diaphragm.

Providing a cartridge housing both a pump chamber for pumping a patients blood to/from a dialyser and a sensor cavity for measuring the pressure of blood pumped results in reduced manufacturing costs and fewer errors in manufacturing due to fewer components being produced.

In one embodiment, the cartridge further comprises a second recess therein, said second recess having a surface, and a flexible diaphragm closing said second recess, the second recess and the flexible diaphragm defining a second pump chamber, said second pump chamber having an inlet and an outlet wherein the flexible diaphragm of the second pump chamber is movable between a first position, separated in use from the surface of the second recess, in which said second pump chamber has a maximum volume, and a second position, substantially adjacent to the surface of the second recess, in which said second pump chamber has a minimum volume.

Provision of two pump chambers on the blood pump cartridge permits the blood pump to be used with single or twin needle configurations. A twin needle configuration is advantageous if greater flow of blood from the patient through the dialyser is required. Use of a twin needle configuration effectively allows for double the blood flow from the patient than use of a single needle configuration.

In one embodiment, the blood pump further comprises a platen, the platen having one or more recesses therein, each recess having a surface, the one or more recesses corresponding substantially in geometry to a recess in the cartridge and being separated therefrom by a flexible diaphragm associated therewith.

Advantageously, the surface(s) associated with the cartridge and the surface(s) associated with the platen provide a positive stop for the flexible diaphragm(s) thus defining the minimum and maximum volumes of the blood pump.

In one embodiment, the flexible diaphragm is pneumatically actuated between said first and second position.

In one embodiment, the flexible diaphragm, when in a first position contacts the surface of the first recess in the cartridge and, when in a second position contacts the surface of the corresponding recess in the platen.

In one embodiment, the first and second pump chambers can be operated in phase with one another.

Operating the first and second pump chambers in phase with one another allows use of the twin needle configuration introduced above.

In another embodiment, the first and second pump chambers can be operated out of phase with one another.

Operating the first and second pump chambers out of phase with one another allows the first pump chamber, for example, to be used to draw blood from the patients artery and to pump blood to the dialyser and allows the second pump chamber, for example, to draw treated blood from the dialyser and return it to the patients vein.

In one embodiment, the blood pump further comprises an inlet valve to the first pump chamber and an inlet valve to the second pump chamber, each of said inlet valves being disposed in an inlet channel.

In one embodiment, the blood pump further comprises an outlet valve from the first pump chamber and an outlet valve from the second pump chamber, each outlet valve being disposed in an outlet channel.

The provision of inlet valves to and outlet valves from each pump chamber permits the first and second pump chambers to be used together, in isolation, in phase with each other or out of phase with each other. Such a configuration permits the blood pump to be used for either single needle or twin needle extracorporeal blood treatment.

In one embodiment, the, or each sensor cavity is hemispherical.

In one embodiment, the, or each sensor cavity further comprises a surface, said surface defining a flat bottom in a recess. Such an arrangement facilitates the reflection of an ultrasound signal or optical signal, enhancing detection fidelity.

An ultrasonic transducer is used to measure a characteristic of the patients blood as it passes through the sensor cavity. The ultrasonic transducer is provided external to the sensor cavity and is in contact with the flexible diaphragm closing the sensor cavity. Provision of a flat bottom in the sensor cavity, internal or external, aids reflection of the ultrasonic waves emitted by the ultrasonic transducer.

In one embodiment, the inlet and outlet of the, or each, sensor cavity are tangential. Provision of tangential inlets and outlets is desirable to reduce stasis of the patients blood within the sensor cavity. The tangential inlets and outlets do not have areas where blood could collect and not circulate and encourage the blood to swirl within the sensor cavity.

In one embodiment, the first pump chamber has a common inlet and outlet and the second pump chamber has a common inlet and outlet.

In one embodiment, the blood pump further comprises an arterial blood inlet, a dialyser blood outlet, a dialyser blood inlet and a venous blood outlet.

In one embodiment, the blood pump comprises three sensor cavities.

In one embodiment, the sensor cavities respectively define an arterial pressure chamber, a dialyser pressure chamber and a venous pressure chamber.

In one embodiment, the blood pump further comprises an inlet channel between the arterial blood inlet and the first pump chamber and the second pump chamber.

In one embodiment, the blood pump further comprises an outlet channel between the dialyser blood outlet and the first pump chamber and the second pump chamber.

In one embodiment, the blood pump further comprises an inlet valve to the first pump chamber and an inlet valve to the second pump chamber, each inlet valve being disposed in the inlet channel.

In one embodiment the arterial pressure chamber is provided downstream of the arterial blood inlet and upstream of the first and second pump chambers.

In one embodiment, the dialyser pressure chamber is provided downstream of the first and second pump chambers and upstream of the dialyser blood outlet.

In one embodiment, the venous pressure chamber is provided downstream of the dialyser blood inlet and upstream of the venous blood outlet.

In one embodiment, the, or each, pump chamber, sensor cavity, valve and channel is provided on a common datum face.

In one embodiment, the blood pump is disposable.

Provision of a disposable blood pump is desirable to remove the need for cleaning of the blood pump after each extracorporeal blood treatment session.

In one embodiment, the flexible diaphragm of the, or each, pump chamber and the, or each, sensor cavity is formed from a single sheet of material.

In one embodiment, the sheet of material defining respective diaphragms is vacuum attached to the cartridge in a region around the, or each, sensor cavity.

Vacuum attaching the flexible diaphragms of the sensor cavities to the cartridge increases reflection from an ultrasonic transducer through the sensor cavities and reduces reflection at the interface between the flexible diaphragm and the cartridge to optimise measurements taken by the transducer by discriminating the ultrasound transducer signal from background noise.

A further aspect of the invention provides a blood pump comprising a cartridge, the cartridge comprising one or more pump chambers and one or more sensor cavities, the, or each pump chamber and the, or each, sensor cavity being provided on a common datum face.

Advantageously, providing each of the features of the blood pump on a common datum face simplifies production of the cartridge and permits the cartridge to be manufactured from a single piece of material. This is desirable as a single cartridge is simpler to load and unload from a hemodialysis machine thus resulting in fewer user errors in using the machine.

Embodiments of the invention will now be described, by way of example only, with reference to the following drawings in which:

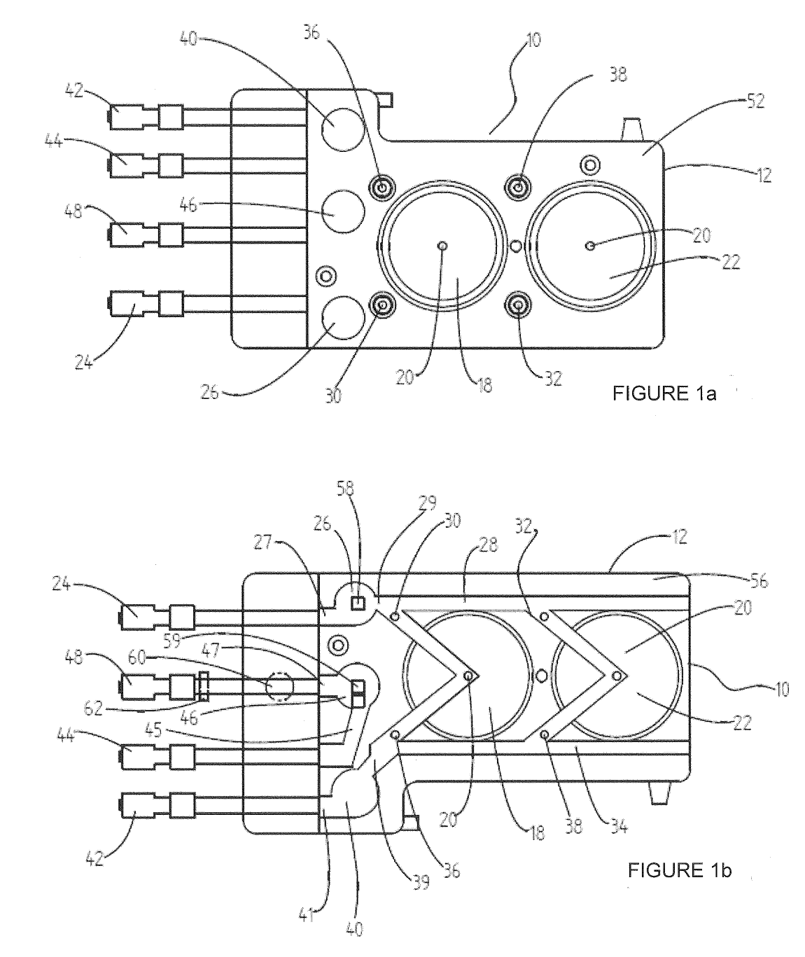

FIG. 1a is a schematic plan of a blood pump according to an embodiment of the invention viewed from one side;

FIG. 1b is a schematic plan of a blood pump according to an embodiment of the invention viewed from the other side;

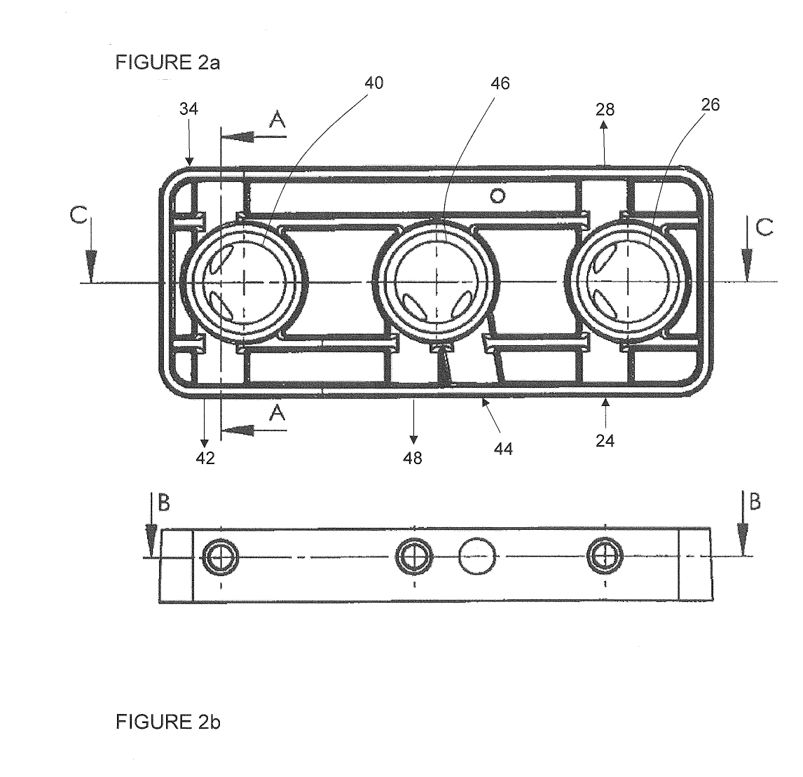

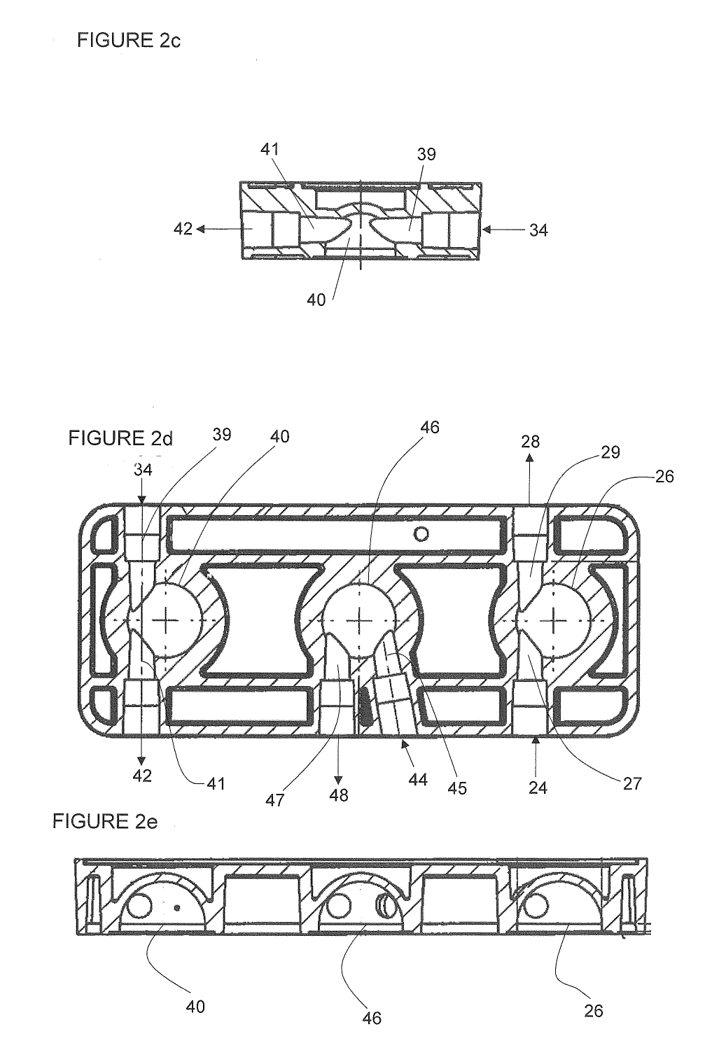

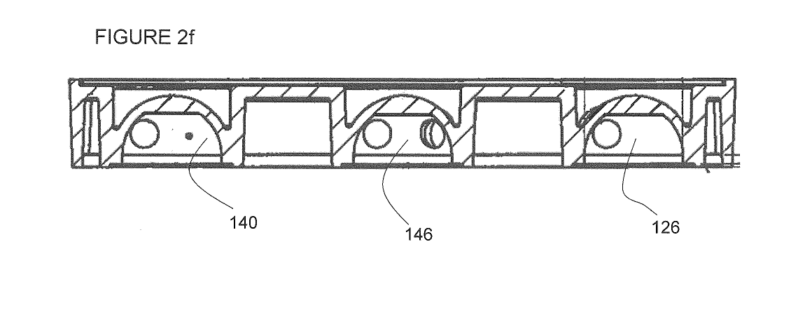

FIG. 2a is an enlarged view of the sensor cavity section of the blood pump of FIG. 1a in isolation (rotated anti-clockwise by 90°);

FIG. 2b is a side view of the sensor cavity section of FIG. 2a;

FIG. 2c is a cross-sectional view of the arterial pressure chamber taken at lines A-A of FIG. 2a;

FIG. 2d is a cross-sectional view of the sensor cavity section mid-plane taken at lines B-B of FIG. 2b;

FIG. 2e is a cross-sectional view of the sensor cavity section mid-plane taken at lines C-C of FIG. 2a;

FIG. 2f is a cross-sectional view of an alternative sensor cavity section represented similarly as FIG. 2e; and

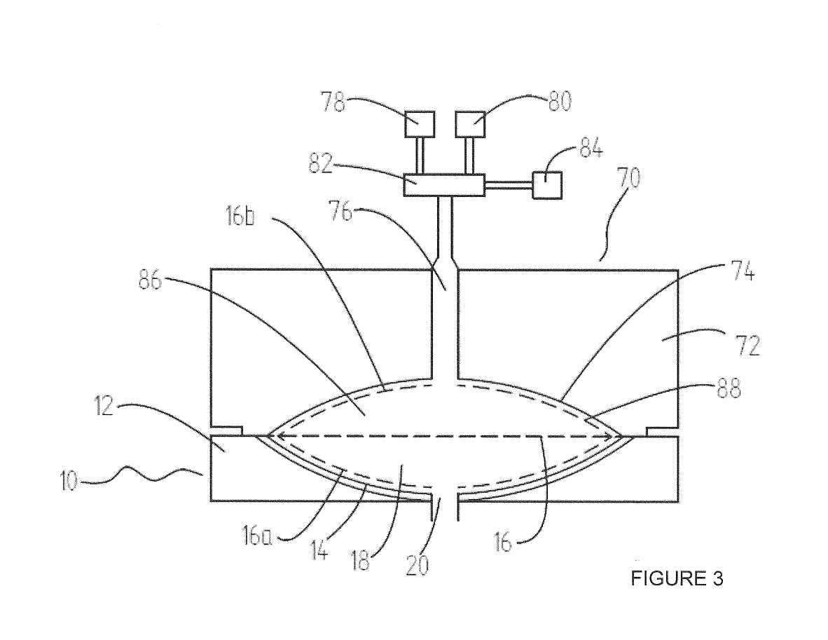

FIG. 3 is a schematic side view of a blood pump according to an embodiment of the invention.

Referring to FIGS. 1 to 3, an embodiment of the invention provides a blood pump 10 comprising a pump cartridge 12 manufactured from a plastic shell and having a concave recessed surface 14 covered by a flexible diaphragm 16. The recessed surface 14 and the flexible diaphragm 16 define a pump chamber 18 of conical, concave or frustroconical shape and having at the apex thereof a common inlet and outlet 20 for both allowing blood to flow into the pump chamber 18 and to be pumped from the pump chamber 18.

In the illustrated example, the cartridge 12 provides two pump chambers 18, 22 but it will be appreciated that the number of pump chambers is not intended to be limited.

Blood is received via a needle from a patients artery through an arterial flow port 24 into a sensor cavity 26 defining an arterial pressure chamber. Blood enters the arterial pressure chamber 26 via an arterial pressure chamber inlet 27. The arterial pressure chamber 26 measures the pressure of blood from the patients artery and the output may be used to distinguish between a correctly placed needle and a dislodged needle. From the arterial pressure chamber 26 blood is received into an inlet channel 28, via an arterial pressure chamber outlet 29. The inlet channel 28 is provided with an inlet valve 30 to the first blood pump chamber 18 and an inlet valve 32 to the second blood pump chamber 22.

The first and second blood pump chambers 18, 22 are selectively operable to run in or out of phase with one another or in or out of phase with further pumps forming part of a hemodialysis machine. From the first and second blood pump chambers 18, 22, blood is pumped to an outlet channel 34, via an outlet valve 36 of the first blood pump chamber 18 and via an outlet valve 38 of the second blood pump chamber 22.

From the outlet channel 34 blood passes to a dialyser through a sensor cavity 40 defining a pre-dialyser pressure chamber. The pre-dialyser pressure chamber 40 has an inlet 39 and an outlet 41. Blood passes out of the cartridge 12 via a dialyser outlet port 42. The pre-dialyser pressure chamber 40 measures blood pressure prior to entering the dialyser to allow the flow rate of blood passing through the dialyser to be calculated.

After passing through the dialyser blood re-enters the cartridge 12 via a dialyser inlet port 44 into a sensor cavity 46 defining a venous pressure chamber. The venous pressure chamber 46 has an inlet 45 and an outlet 47. The venous pressure chamber 46 measures blood pressure blood prior to returning to the patients vein from the cartridge 12 via a venous outlet port 48. The venous pressure chamber 46 sends a signal to a modulated control valve 82 in response to variations in the venous return blood pressure caused by the patient moving around.

Each sensor cavity 26, 40, 46 comprises a concave recess covered by a flexible diaphragm to define the respective hemispherical pressure chambers. The hemispherical pressure chambers thereby present an upturned bowl shape, which minimizes the surface area to volume ratio, and precludes the provision of corners which would otherwise provide stagnation points for the flow. The inlets and outlets from each sensor cavity are arranged as will be described below, to further minimise stasis in the blood. Stasis occurs when flow of fluid is interrupted by an obstruction. Blood entering a sensor cavity through an inlet swirls within the sensor cavity until it exits through an outlet.

With reference to FIGS. 2a to 2e, the sensor cavities 26, 40, 46 define respective hemispherical pressure chambers, bounded on one side by the flexible diaphragm. Each inlet enters the pressure chambers at an elliptical orifice, as the cylindrically shaped channels 24, 34, 44 meet the hemispherical pressure chamber wall. Thus their respective inlets 27, 39, 45 provide a shear profile across the inlet orifice in the blood flow as it enters the cavities which enhances mixing.

Furthermore, in the cases of the arterial pressure chamber 26 and pre-dialyser pressure chamber 40, the proximity of their respective inlets 27, 45 to the pressure chamber wall provides a surface drag, decelerating the blood flow asymmetrically, also enhancing mixing.

In the case of the venous pressure chamber 46, the inlet 45 and outlet 47 are mis-aligned, with the inlet 44 being non-tangential with the venous pressure chamber wall to prevent the blood flow from simply attaching to the wall and exiting the venous pressure chamber 46 at the outlet 47 following a “U-turn” flow path, forcing the blood flow to impinge upon the cavity wall opposite the inlet 45.

The unsteady flow pattern created by the arrangement of the cavities 26, 40, 46 and their respective inlet and outlets 27, 39, 45, 29, 41, 47, coupled with the pulsible nature of the membrane pump referred to above minimises stasis and maximises mixing of the blood in a spacially compact arrangement.

Although the illustrated embodiment is described having an arterial pressure chamber 26, venous pressure chamber 46 and pre-dialyser pressure chamber 40, the provided sensor cavities are not intended to be limited for such purposes. For example, the sensor cavities can be used for detecting pressure, bubbles, blood, hematocrit and urea clearance, for example by means of known apparatus and techniques. Additionally, although three sensor cavities are shown in the illustrated embodiment, it will be appreciated that more or less than three sensor cavities could be provided.

In an alternative sensor cavity (see FIG. 2f) the hemispherical pressure chambers are flattened so as to define two parallel surfaces, one surface being the flexible diaphragm and the other surface being a flat bottom in the recess. Similar reference numbers are used to identify similar features, prefixed with a ‘1’ to denote that those features are of the alternate sensor cavity.

The arterial pressure chamber 46, of the illustrated embodiment, comprises a pressure transducer 58 for controlling a modulated valve 82 (see FIG. 3) provided on a hemodialysis machine, as described below. The pressure transducer 58 provides feedback to a controller 84 to prevent excessive vacuum being used to draw blood into the pump chamber(s) 18, 22, as described below.

The pump chambers, sensor cavities and valves all share a common datum face 52 covered by a single, common flexible sheet of material defining the respective pump chamber and sensor cavity diaphragms. In the illustrated embodiment, the inlet and outlet channels 28, 34 are provided on the opposing face 56 to the common datum face 52 of the cartridge and are closed by a second single, common flexible sheet of material (not shown).

In an alternative embodiment the inlet and outlet channels 28, 34 are disposed between and sealed by both flexible sheets of material.

The flexible sheet of material sealing the common datum surface 52 is held against the cartridge 12 by vacuum around each sensor cavity. The flexible sheet of material sealing the common datum surface 52 is attached by adhesive to the cartridge 12 at its periphery and around each pump chamber and valve to define the respective diaphragms. The flexible sheet of material (not shown) sealing the surface 56 opposing the common datum surface 52 is attached to the cartridge 12 by adhesive.

The cartridge 12 contains a thrombus trap 59 and a bubble trap 60, of known types, moulded therein in the form of flow through cells. The thrombus trap 59 could be located, for example, in a sensor cavity. The bubble trap 60 comprises a blood inlet (not shown), a blood outlet (not shown) and a vent (not shown) to the common datum face 52 side of the cartridge 12.

A venous clamp 62 is attached to the venous return line to the patient. Upon activation of a hemodialysis machine safety system, the venous clamp 62 is applied to prevent further flow of blood to the patient. In use, the safety system is adapted to also de-activate the blood pump chambers 18, 22 to prevent further blood being pumped from the patient.

With reference to FIG. 3, the cartridge 12 abuts a pump driver 70 (See FIG. 3) comprising a platen 72 having a recessed surface 74 therein and a fluid port 76. In use, the platen 70 is kinematically located against the cartridge 12 to sealingly engage with the cartridge 12 such that the recessed surface 74 and the flexible diaphragm 16 define a drive chamber 86. A sensor (not shown) detects whether the cartridge 12 is located correctly and generates an alarm signal if the cartridge 12 is incorrectly located. The cartridge 12 is held against the platen 70 by a door (not shown) and a sensor (not shown) detects whether the door is open or closed.

The fluid port 76 is connectable with a source of positive fluid pressure 78 and a negative source of fluid pressure 80 via a modulated valve 82, controlled by the controller 84 to allow fluid to flow into or out of the drive chamber 86.

The modulated valve 82 is a proportional valve having a variable sized orifice therein, the valve being controllable to change the size of the orifice, thereby controlling the flow of fluid therethrough.

The sources of positive and negative fluid pressure 78, 80 include a pressure pump and a vacuum pump respectively. When the modulated valve 82 is operated to allow fluid to flow into the drive chamber 86 from the source of positive fluid pressure 78, the flexible diaphragm 16a moves towards the recessed surface 14 and any blood that is in the pump chamber 18, 22 is pumped out of the common inlet and outlet 20. When the modulated valve 82 is operated to allow fluid to flow out of the drive chamber 86 to the source of negative fluid pressure 80, the flexible diaphragm 16b is moved away from the recessed surface 14 towards surface 74 and blood is drawn into the pump chamber 18, 22 from the common inlet and outlet 20.

In order to pump blood through the pump chambers 18, 22, the common inlet and outlet 20 of each pump 18, 22 has an inlet valve 30, 32 and an outlet valve 36, 38 associated therewith. In use, when the modulated valve 82 is operated to allow fluid into the drive chamber 86 from the source of positive fluid pressure 78, the inlet valve 30, 32 of the pump chamber 18, 22 is closed and the outlet valve 36, 38 of the pump chamber 18, 22 is open so that the blood within the pump chamber 18, 22 exits the common inlet and outlet 20 via the outlet valve 36, 38 of the pump chamber 18, 22.

When the modulated valve 82 is operated to allow fluid to flow out of the drive chamber 86 to the source of negative fluid pressure 80, the inlet valve 30, 32 of the pump chamber 18, 22 is opened and the outlet valve 36, 38 of the pump chamber 18, 22 is closed such that blood is drawn into the pump chamber 18, 22 through the common inlet and outlet 20 via the open inlet valve 30, 32 of the pump chamber 18, 22.

The inlet valves 30, 32 and outlet valves 36, 38 of the pump chambers 18, 22 are, in use, configured to operate to minimise pressure spikes in the patients blood.

When changing from filling to emptying the pump chamber 18, 22, the inlet valve 30, 32 of the pump chamber 18, 22 is closed and the outlet valve 36, 38 of the pump chamber 18, 22 is opened before flow of blood commences from the pump chamber. Opening the outlet valve 36, 38 of the pump chamber 18, 22 before flow of blood commences from the pump chamber 18, 22 ensures that there is no resistance against the flow of blood out of the common inlet and outlet 20. The outlet valve 36, 38 of the pump chamber 18, 22 is not opened instantaneously. Opening the outlet valve 36, 38 of the pump chamber 18, 22 at the same time as flow of blood commences from the pump chamber 18, 22 would create a positive pressure spike within the blood and cause rupturing of red blood cells.

When changing from emptying to filling the pump chamber 18, 22, the outlet valve 36, 38 of the pump chamber 18, 22 is closed and the inlet valve 30, 32 of the pump chamber 18, 22 is opened before flow of blood commences to the pump chamber 18, 22. Opening the inlet valve 30, 32 of the pump chamber 18, 22 before flow of blood commences to the pump chamber 18, 22 ensures that there is no resistance against the flow of blood into the common inlet and outlet 20 of the pump chamber 18, 22. The inlet valve 30, 32 of the pump chamber 18, 22 is not opened instantaneously. Opening the inlet valve 30, 32 of the pump chamber 18, 22 at the same time as flow of blood commences into the pump chamber 18, 22 would create a negative pressure spike within the blood and cause rupturing of red blood cells.

The inlet valves 30, 32 and the outlet valves 36, 38 of the pump chambers 18, 22 may be operated such that when the flexible diaphragm 16 of a pump chamber 18, 22 is at one extremity of its travel, either adjacent the concave recess 14 or adjacent the recessed surface 72, the valve of the pump chamber 18, 22, that is opening opens before the valve of the pump chamber 18, 22 that is closing closes, i.e. both valves of the pump chamber are momentarily open.

For example, when positive pressure is applied to the flexible diaphragm 16 it travels in the direction towards the concave recess 14, displacing blood through the common inlet and outlet 20 via the open outlet valve 36, 38 of the pump chamber 18, 22.

Once the flexible diaphragm 16a has reached the concave recess 14, the inlet valve 30, 32 of the pump chamber 18, 22 is first opened, the outlet valve 36, 38 of the pump chamber 18, 22 is then closed and then the modulated valve 82 is operated to allow fluid to flow out of the drive chamber 86 such that the flexible diaphragm 16 starts to move in the direction away from the concave recess 14 and towards the recessed surface 72.

In a similar manner, when the diaphragm 16b reaches the extremity of its travel adjacent the recessed surface 72, the outlet valve 36, 38 of the pump chamber 18, 22 is first opened, the inlet valve 30, 32 of the pump chamber 18, 22 is then closed, and the modulated valve 82 is then operated to allow fluid to flow into the drive chamber 86 such that the flexible diaphragm 16b starts to move in the direction away from the recessed surface 72 and towards the concave recess 14.

Although the blood pump(s) is/are described with reference to a pump chamber 18, 22 having a single common inlet and outlet 20, each pump chamber 18, 22 could also be provided with two inlet ports and two outlet ports while having the same effect in minimising stasis within the patients blood. Each pump chamber 18, 22 could also be provided with more than two inlet ports and a corresponding number of outlet ports.

In another embodiment, the blood pump is a disposable blood pump comprising a disposable pump cartridge.

The embodiments of the invention, described with reference to the figures, are examples only and do not exclude variations therefrom from the scope of the invention as defined by the claims.

1. A blood pump comprising:
a cartridge, the cartridge comprising a first recess therein, said first recess having a surface, and a flexible diaphragm closing said first recess, the first recess and the flexible diaphragm defining a first pump chamber, wherein the flexible diaphragm of the first pump chamber is movable between a first position, separated in use from the surface of the first recess, in which said first pump chamber has a maximum volume, and a second position, substantially adjacent to the surface of the first recess, in which said first pump chamber has a minimum volume; and
a pump driver arranged to interface with the cartridge, said pump driver operable to move the flexible diaphragm of the first pump chamber in a first direction into said first recess to, in use, pump blood from the first pump chamber, and to move the flexible diaphragm of the first pump chamber in a second direction away from the first recess to, in use, draw blood into said first pump chamber;
wherein, the cartridge further comprises one or more sensor cavities defined by respective recesses in the cartridge, the, or each, recess being closed by a flexible diaphragm.

2. The blood pump according to claim 1, wherein the cartridge further comprises a second recess therein, said second recess having a surface, and a flexible diaphragm closing said second recess, the second recess and the flexible diaphragm defining a second pump chamber, wherein the flexible diaphragm of the second pump chamber is movable between a first position, separated in use from the surface of the second recess, in which said second pump chamber has a maximum volume, and a second position, substantially adjacent to the surface of the second recess, in which said second pump chamber has a minimum volume.

3. The blood pump according to claim 1, wherein the, or each, sensor cavity further comprises an inlet and an outlet.

4. The blood pump according to claim 1, wherein the, or each, sensor cavity is hemispherical.

5. The blood pump according to claim 4, wherein the recess defining the, or each, sensor cavity further comprises a surface, said surface defining a flat bottom in said recess.

6. The blood pump according to claim 3, wherein the inlet and the outlet of the, or each, sensor cavity is tangential.

7. The blood pump according to claim 3, wherein the inlet of the, or each, sensor cavity is offset from the respective outlet of the, or each, sensor cavity.

8. The blood pump according to claim 3, wherein one of the inlet or the outlet of the, or each, sensor cavity is offset radially with respect to the respective sensor cavity.

9. The blood pump according to claim 2, wherein the first pump chamber has a common inlet and outlet and the second pump chamber has a common inlet and outlet.

10. The blood pump according to claim 2, wherein the blood pump further comprises an arterial blood inlet, a dialyser blood outlet, a dialyser blood inlet and a venous blood outlet.

11. The blood pump according to claim 2, wherein the one or more sensor cavities comprise three sensor cavities.

12. The blood pump according to claim 11, wherein the three sensor cavities respectively define an arterial pressure chamber, a dialyser pressure chamber and a venous pressure chamber.

13. The blood pump according to claim 10, wherein the arterial blood inlet is in fluid connection with the first pump chamber and the second pump chamber.

14. The blood pump according to claim 10, wherein the dialyser blood outlet is in fluid connection with the first pump chamber and the second pump chamber.

15. The blood pump according to claim 14, wherein the blood pump further comprises an inlet channel between the arterial blood inlet and the first pump chamber and the second pump chamber.

16. The blood pump according to claim 14, wherein the blood pump further comprises an outlet channel between the dialyser blood outlet and the first pump chamber and the second pump chamber.

17. The blood pump according to claim 15, wherein the blood pump further comprises an inlet valve to the first pump chamber and an inlet valve to the second pump chamber, each inlet valve being disposed in the inlet channel.

18. The blood pump according to claim 16, wherein the blood pump further comprises an outlet valve from the first pump chamber and an outlet valve from the second pump chamber, each outlet valve being disposed in the outlet channel.

19. The blood pump according to claim 12, wherein
the blood pomp further comprises an arterial blood inlet; and
the arterial pressure chamber is provided downstream of the arterial blood inlet and upstream of the first and second pump chambers.

20. The blood pump according to claim 12, wherein
the blood pump further comprises a dialyser blood outlet; and
the dialyser pressure chamber is provided downstream of the first and second pump chambers and upstream of the dialyser blood outlet.

21. The blood pump according to claim 12, wherein
the blood pump further comprises a dialyser blood inlet and a venous blood outlet; and
the venous pressure chamber is provided downstream of the dialyser blood inlet and upstream of the venous blood outlet.

22. The blood pump according to claim 1, wherein the first pump chamber and the one or more sensor cavities are provided on a common datum face.

23. The blood pump according to claim 1, wherein the blood pump is disposable.

24. The blood pump according to claim 1, wherein the flexible diaphragms of the first pump chamber and the one or more sensor cavities are formed from a single sheet of material.

25. The blood pump according to claim 24, wherein the sheet of material defining respective diaphragms is vacuum attached to the cartridge in a region around the, or each, sensor cavity.

26. The blood pump according to claim 1, the blood pump further comprising a bubble trap.

27. The blood pump according to claim 26, wherein the bubble trap is downstream of the first pump chamber.

28. The blood pump according to claim 1, wherein the blood pump further comprises a thrombus trap.

29. The blood pump according to claim 28, wherein the thrombus trap is provided within one of the one or more sensor cavities.

30. A blood pump comprising:
a cartridge;
the cartridge comprising one or more pump chambers and one or more sensor cavities;
the, or each pump chamber and the, or each, sensor cavity being provided on a common datum face of the cartridge.

31. The blood pump according to claim 30, wherein the cartridge further comprises an inlet valve associated with the, or each, pump chamber and an outlet valve associated with the, or each, pump chamber, each said inlet valve and each said outlet valve being provided on the common datum face of the cartridge.

32. The blood pump according to claim 30, wherein the cartridge further comprises an inlet channel to the, or each pump chamber and an outlet channel from the, or each, pump chamber, said net channel and said outlet channel being provided on the common datum face of the cartridge.

33. (canceled)
